(19) United States
(12) Patent Application Publication
(10) Pub. No.: US 2017/0182309 A1
(43) Pub. Date: Jun. 29, 2017

(54) TEMPLATE FOR TRIGEMINAL NEUROSTIMULATION

(71) Applicant: NeuroSigma, Inc., Los Angeles, CA (US)

(72) Inventors: Leon Akchian, Los Angeles, CA (US); David Hayes, Los Angeles, CA (US)

(21) Appl. No.: 15/462,111

(22) Filed: Mar. 17, 2017

Related U.S. Application Data
(63) Continuation of application No. PCT/US2015/050804, filed on Sep. 17, 2015.
(60) Provisional application No. 62/051,853, filed on Sep. 17, 2014.

(51) Int. Cl. A61N 1/04; A61N 1/36

(52) U.S. Cl. CPC A61N 1/0456; A61N 1/36014

(57) ABSTRACT

A template is provided that includes two apertures spaced apart by the expected spacing between a pair of ophthalmic nerves on a patient's forehead. The template may thus be advantageously used to apply cutaneous electrodes to a patient's forehead for trigeminal nerve stimulation without clinical training.

### CROSS-REFERENCE TO RELATED APPLICATION

This application is a continuation of International Patent Application No. PCT/US2015/050804, filed Sep. 17, 2015, and entitled “TEMPLATE FOR TRIGEMINAL NEUROSTIMULATION,” which claims the benefit of U.S. Provisional Application No. 62/051,853, filed Sep. 17, 2014, and entitled “TEMPLATE FOR TRIGEMINAL NEUROSTIMULATION,” which are hereby incorporated by reference in their entirety.

### TECHNICAL FIELD

The present disclosure relates to neurostimulation, and more specifically to the stimulation of cranial nerves using electrodes placed through a template.

### BACKGROUND

Neurological disorders such as seizure disorders are usually treated with medication. However, there are patients who are not helped by medication—they may not be able to tolerate the side effects or the medication itself is not efficacious for their particular disorder. This is a significant problem in that seizure disorders can be life threatening. Moreover, the quality of life for victims of severe epilepsy can be severely impacted. Neuropsychiatric disorders such as depression and ADHD are also typically treated with medications that have deleterious side effects and lack of efficacy. To offer patients relief that medication alone cannot deliver, various neurostimulation methods have been developed. For example, vagus nerve stimulation (VGS) has been shown to be therapeutically useful. Similarly, deep brain stimulation (DBS) and responsive neurostimulation (RNS) approaches are known to have efficacy. But these neurostimulation techniques are invasive as they require surgical implantation of electrodes. Thus, these techniques are relatively expensive and involve the dangers associated with the surgical implantation of the electrodes.

To provide neurostimulation without the invasive dangers of prior art techniques, an alternative neurostimulation therapy has been developed that involves trigeminal nerve stimulation (TNS). For example, a cutaneous embodiment of TNS involves the transcutaneous stimulation of the supraorbital nerves and/or the supratrochlear nerves in the forehead. Like other cranial nerves, the supraorbital and supratrochlear nerves arise through foramina or notches in the skull. The supraorbital nerve arises from the supraorbital foramen or notch above the orbit. Since one has two eyes, there are thus two supraorbital nerves that ascend vertically toward the scalp from their respective foramen. The supratrochlear nerve is medial with regard to the supraorbital. But it also then ascends vertically towards the hairline. There are thus two supratrochlear nerves, each arising from its respective orbit. A supraorbital nerve and supratrochlear nerve pair associates with each orbit. The forehead is thus an ideal location to stimulate the trigeminal nerve in that the supraorbital nerve and supratrochlear nerve associated with each orbit are located medially on the forehead. The skin and fascia over the forehead is relatively thin such that the supratrochlear and supraorbital nerves are readily stimulated transcutaneously.

One approach to stimulate the supratrochlear and supraorbital nerves requires a clinician to palpate for the supraorbital notch or foramen so that a suitable electrode can be applied adjacent the notch. The electrode would be sized so that it would cover not only the trunk of the supraorbital as it arises from its foramen but also the trunk of the corresponding supratrochlear nerve. To provide bilateral stimulation, the clinician would also palpate for the remaining supraorbital notch and apply another electrode accordingly. Although such an approach provides advantageous neurostimulation for treatment of disorders without invasive implantations or deleterious pharmaceutical side effects, the treatment is burdened by the need for expert application of the electrodes. For example, if a lay person applies the electrodes in this fashion and locates the electrodes too laterally on the forehead, the resulting bilateral current excited between the two electrodes may penetrate to the brain. Thus, the application of electrodes in this fashion required medical expertise, which greatly increases costs as the patient must visit a medical facility daily for chronic treatments.

Accordingly, there is a need in the art for improved TNS electrode application techniques.

### SUMMARY

A method for trigeminal nerve stimulation is providing that includes positioning an electrode template on a patient so that a first aperture in the electrode template is over a supraorbital nerve on one side of a patient's forehead and so that a second aperture in the electrode template is over a supraorbital nerve on an opposing side of the patient's forehead. The method also includes applying a first cutaneous electrode through the first aperture in the electrode template so that the first cutaneous electrode is cutaneously applied over the supraorbital nerve on the one side of a patient's forehead as well as applying a second cutaneous electrode through the second aperture in the electrode template so that the second cutaneous electrode is cutaneously applied over the supraorbital nerve on the opposing side of the patient's forehead. Finally, the method includes driving a current between the first cutaneous electrode and the second cutaneous electrode at specified operational parameters to provide the trigeminal nerve stimulation.

A template for placement of electrodes for trigeminal nerve stimulation is also provided. The template includes: a planar template body; a first aperture at a first lateral edge of the template body; and a second aperture at an opposing second lateral edge of the template body, wherein the template body is laterally elongated from the first lateral edge to the second lateral edge such that the first aperture is configured for cutaneous placement of a first electrode over a supraorbital nerve on one side of a patient's face forehead and such that the second aperture is configured for cutaneous placement of a second electrode comprising over a remaining supraorbital nerve on an opposing side of the patient's forehead

### BRIEF DESCRIPTION OF THE DRAWINGS

The present disclosure, both as to its organization and manner of operation, may be understood by reference to the following description, taken in connection with the accompanying drawings, in which:

FIG. 1A shows a patient receiving TNS therapy through electrodes placed using a template in accordance with an embodiment of the disclosure.

FIGS. 1B and 1C illustrate the location of several branches (nerves) of the trigeminal nerve and the location of the major foramina for the superficial branches of the trigeminal nerve;

FIG. 2 illustrates an example template for the placement of cutaneous electrodes to provide TNS therapy.

### DETAILED DESCRIPTION

As discussed above, there is a need in the art for improved TNS therapy methods. To satisfy this need in the art, trigeminal neurostimulation (TNS) techniques using electrodes placed through a template are disclosed.

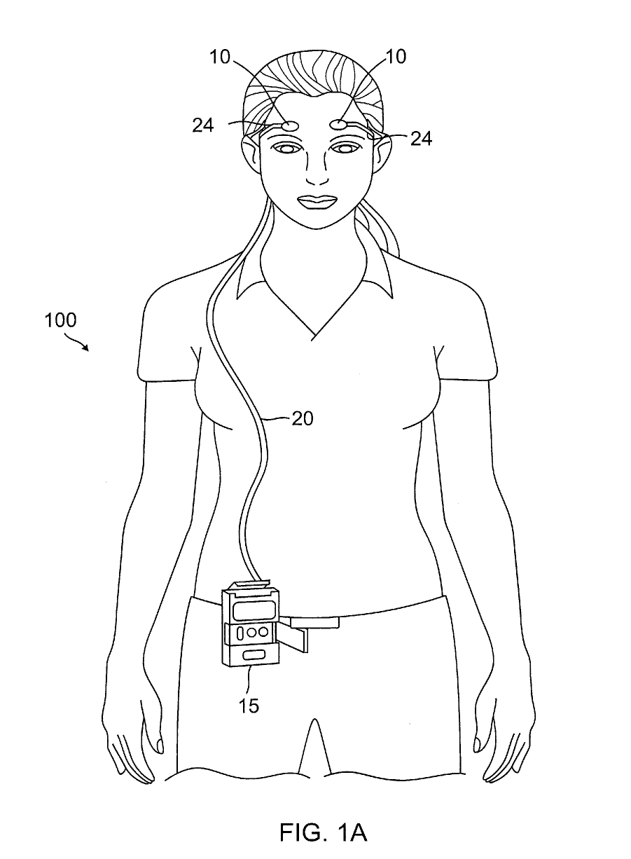

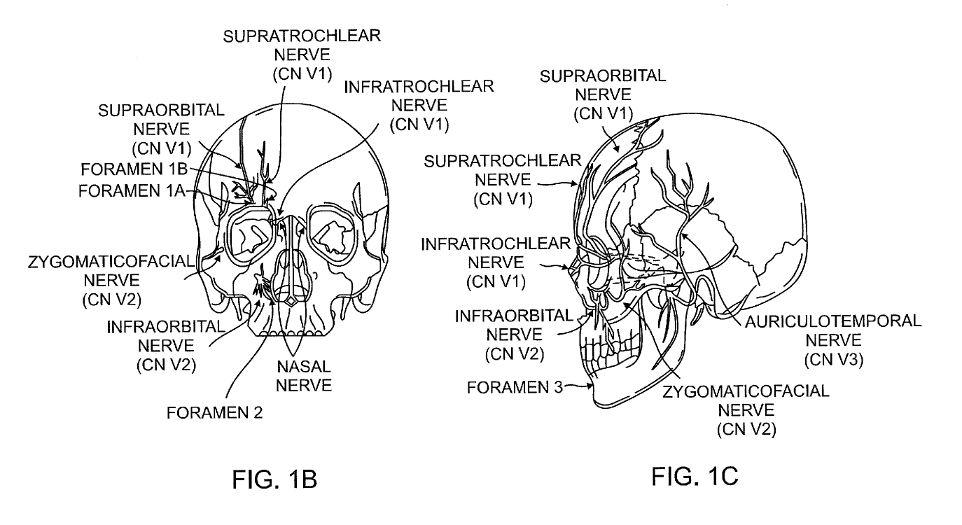

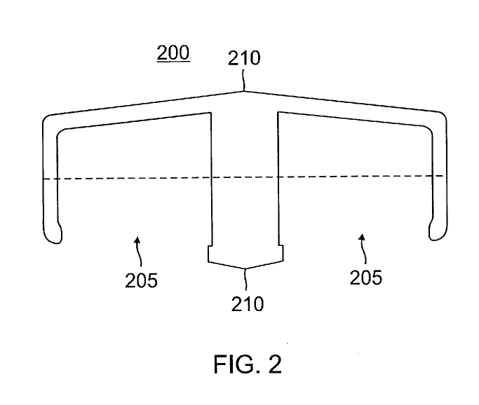

FIG. 1A illustrates a patient having cutaneous electrodes 10 placed using a template 200 shown in FIG. 2. To provide efficacious trigeminal neurostimulation therapy without the need for daily medical facility visits, the patient first medially centers template 200 across their forehead. Template 200 is constructed with apertures 205 within which the patient (or a clinician) applies electrodes 10. In this fashion, each aperture 205 receives a corresponding electrode 10. Due to the configuration of template 200, the placed electrodes 10 are then positioned over the supraorbital and/or supratrochlear trunks (not illustrated). To ensure the maximum coverage or stimulation of each supraorbital and supratrochlear nerve trunk, the patent may be instructed to align an inferior edge of template 200 just above their orbital arches. The anatomy for these nerve trunks will now be discussed.

With reference to FIGS. 1B and 1C, the trigeminal nerve is the largest cranial nerve and has extensive connections with the brainstem and other brain structures. The trigeminal nerve, also named the fifth cranial nerve or “CN V,” has three major sensory branches over the face, all of which are bilateral, and highly accessible. The ophthalmic nerve is frequently referred to as the V₁ division and includes the supraorbital and supratrochlear nerves that supply sensory information about pain, temperature, and light touch to the skin of the forehead, the upper eyelid, the anterior part of the nose, and the eye. The V₂ division includes the infraorbital and maxillary nerves. The infraorbital branch supplies sensory information about pain, temperature, and light touch sensation to the lower eyelid, cheek, and upper lip. Finally, the V₃ division includes the auriculotemporal, lingual, and inferior alveolar branches of the mandibular nerves. The inferior alveolar branch supplies similar sensory modalities to the skin of the lower face (e.g. jaw and tongue) and lips.

These branches exit the skull through three groups of foramina or notches, as shown in FIGS. 1b and 1C. The supraorbital and supratrochlear nerves exit at foramina 1. In particular, the foramen (or notch) for the supratrochlear nerve is approximately 2.1-2.6 cm from the nasal midline (in adults), and is located immediately above the orbital ridge that is located below the eyebrow. The supratrochlear foramen is indicated as foramen 1B. In contrast, the foramen (or notch) for the supraorbital nerve is located more laterally from the nasal midline: e.g., approximately 3.2 cm from the nasal midline in adults. This foramen is indicated as foramen 1A. The infraorbital branch or maxillary nerve exits at foramen 2, approximately 2.4-3.0 cm from the nasal midline (in adults) and the mentalis nerve exits at foramen 3, approximately 2.0-2.3 cm from the nasal midline (in adults). Other sensory branches, including the zygomaticofacial, zygomaticoorbital, zygomaticotemporal, and auriculotemporal, arise from other foramina.

Fibers from the three major branches join together to form the trigeminal ganglion. From there, fibers ascend into the brainstem at the level of the pons to synapse with the main sensory nucleus of the pons, the mesencephalic nucleus of V, and the spinal nucleus and tract of V. Pain fibers descend in the spinal nucleus and tract of V, and then ascend to the ventral posterior medial nucleus (VPM) of the thalamus, and then project to the cerebral cortex. Light touch sensory fibers are large myelinated fibers, which ascend to the ventral posterior lateral (VPL) nucleus of the thalamus, and also project to the cerebral cortex. Afferent sensory fibers project from the trigeminal nuclei to the thalamus and the cerebral cortex.

With regard to a given supraorbital arch (either the left or right side of the forehead), the corresponding supraorbital nerve and the adjacent supratrochlear nerve are referred to herein as an “ophthalmic nerve pair.” In this fashion, the ambiguity that results from referring to just the supraorbital nerve (or the supratrochlear) as the “ophthalmic” nerve is avoided.

Referring again to FIG. 2, in an embodiment in which template 200 is constructed for bilateral stimulation, an electrode 10 will thus be on located above each orbit and over the supraorbital notches/foramina such that a current pulse transmitted between the electrodes 10 will conduct across the supraorbital and supratrochlear nerve fibers as they arise from their respective orbits. Moreover, these nerve branches are relatively shallow with regard to the forehead skin surface and thus readily stimulated by electrodes 10.

A pulse generator 15 drives electrodes 10 through a common cable 20 that bifurcates into individual leads 24 for driving electrodes 10. It is important that a patient be able to correctly position each electrode 10 so that the appropriate nerves are stimulated without the risks of current penetration to the brain. Because a patient can readily position template 200 medially on their forehead using a landmark such as their nasal midline, the patient needs no knowledge of anatomy in that regard yet they are positioning the template 200 in an advantageous location for TNS therapy. Studies have shown that TNS carried out with properly-placed electrodes are significantly more efficacious than the use of conventional VNS. Yet TNS is far less invasive, has much fewer risks, and considerably lower cost than VNS.

Referring back to FIGS. 1A and 1B, suppose that there is an electrode 10 over or lateral to each supraorbital nerve. A current driven through one of the contacts into the remaining electrode will thus pass across not only the supraorbital nerves but also across the supratrochlear nerves. One can see that each supraorbital nerve arises from its foramen just medially to the center of each supraorbital ridge. Referring again to FIG. 1A, cutaneous TNS excitation is thus readily achieved by lay people in that template 200 is readily centered on the forehead such that each aperture 205 is positioned over the corresponding ophthalmic nerve pair. The width of each electrode 10 may be such that it is greater than the expected spacing between the supraorbital nerve and the supratrochlear nerve in a given ophthalmic nerve pair. This is quite advantageous as compared to prior art TNS approaches in which individual contacts were positioned by palpating for the supraorbital notch or foramen and attaching an electrode over or above the foramen. Such an individual contact placement is problematic in that a lay person may not attach the contacts properly, which may result in excessive current exposure such that the brain itself receives appreciable currents. But with template 200, the lay person may readily center its midline with the nasal midline. Since the apertures 205 are positioned apart so that an electrode 10 placed within the aperture stimulates the underlying ophthalmic nerve pair, the problems and dangers of prior art individual electrode application are avoided.

As seen in FIG. 2, template 200 may include a midline alignment feature on either of its longitudinal borders to assist in the alignment of template 200 with the nasal midline. For example, a midline alignment feature such as a convex angle 210 (e.g., an angle of 168 degrees) may be defined by the bottom and top longitudinal edges of template 200. Alternatively, only one of the bottom or top edges of template 200 may include such an alignment feature. Apertures 205 are separated by approximately 14 mm. Given the chevron shaping resulting from convex angles 210, each aperture 205 narrows by 12 degrees from a medial edge of a 27 mm to a lateral edge of 20 mm (with regard to the template midline) over a width of 31.5 mm. The lateral edge of each aperture 205 is thus 38.5 mm from the nasal midline. Such an aperture spacing assures that each aperture 205 is positioned over an ophthalmic nerve pair so that an electrode placed within aperture 205 will then stimulate both the supraorbital nerve and the supraorbital nerve in the corresponding ophthalmic nerve pair for the vast bulk of the adult population. But some adults will require even a greater aperture/electrode width such as 34 mm to assure that the supraorbital nerves receive adequate stimulation. It will be appreciated that many alternative embodiments exist for template 200 with regard to inclusion of suitable apertures and midline alignment features. Template 200 is quite advantageous in that a lay person may readily center it about their nasal midline. Since the apertures 205 are spaced apart so that each aperture is positioned over an ophthalmic nerve pair on opposing sides of the forehead, a lay person may readily apply cutaneous electrodes through apertures 205 such that the applied electrodes are properly positioned over the ophthalmic nerves without requiring clinical expertise.

Referring again to FIG. 1A, once a patient or clinician has placed template 200 appropriately on the forehead and applied the electrodes 10 within apertures 205, template 200 may then be removed so that TNS therapy may ensue. In various embodiments, the stimulation is delivered at a specific pulse width or range of pulse widths (or pulse duration). The stimulation can be set to deliver pulse widths in any range within a lower limit of about 10 microseconds and an upper limit of about 3 seconds. In various embodiments, the stimulation can be set to deliver pulse widths in the range greater than and/or less than one or more of 50 μs, 60 μs, 70 μs, 80 μs, 90 μs, 100 μs, 125 μs, 150 μs, 175 μs, 200 μs, 225 μs, 250 μs, up to 500 μs. Those of skill in the art will recognized that one or more of the above times can be used as a border of a range of pulse widths

In some embodiments, the stimulation amplitude is delivered as a voltage or current controlled stimulation. In other embodiments it can be delivered as a capacitive discharge. In various embodiments, the current amplitude can be in any range within a lower limit of about 300 μA and an upper limit of about 30 mA-35 mA, depending on the surface area of the electrodes, inter-electrode distance, the branch(es) stimulated, and the modeling data as described above. In various embodiments, the amplitude can be in a range greater than and/or less than one or more of 50 μA, 75 μA, 100 μA, 125 μA, 150 μA, 175 μA, 200 μA, 225 μA, 250 μA, 275 μA, 300 μA, 325 μA, 350 μA, 375 μA, 400 μA, 425 μA, 450 μA, 475 μA, 500 μA, 525 μA, 550 μA, 575 μA, 600 μA, 625 μA, 650 μA, 675 μA, 700 μA, 725 μA, 850 μA, 875 μA, 900 μA, 925 μA, 950 μA, 975 μA, 1 mA, 2 mA, 3 mA, 4 mA, 5 mA, 6 mA, 7 mA, 8 mA, 9 mA, 10 mA, 11mA, 12 mA, 13 mA, 14 mA, 15 mA, 16 mA, 17 mA, 18 mA, 19 mA and 20 mA. Those of skill in the art will recognize that one or more of the above amplitudes can be used as a border of a range of amplitudes.

In various embodiments, the stimulation can be delivered at one or more frequencies, or within a range of frequencies. The stimulation can be set to be delivered at frequencies in any range within an upper limit of about 500 Hz and a lower limit of about 10 Hz. In various embodiments, the stimulation can be set to be delivered at frequencies less than, and/or greater than one or more of 50 Hz, 45 Hz, 40 Hz, 35 Hz, 30 Hz, 25 Hz, 20 Hz, 15 Hz, or 10 Hz. In various embodiments, the stimulation can be set to be delivered at frequencies greater than, and/or less than, one or more of 20Hz, 30Hz, 40Hz, 50 Hz, 60 Hz, 70 Hz, 80 Hz, 90 Hz, 100 Hz, 125 Hz, 150 Hz, up to 300 Hz. Those of skill in the art will recognize that one or more of the above frequencies can be used as a border of a range of frequencies.

In various embodiments, the stimulation is delivered at a specific duty cycle or range of duty cycles within a range from 100% down to about 5%. The duty cycle is defined with regard to a duty cycle period. In each duty cycle period, the current is pulsed during an on portion of the duty cycle period and not pulsed during a remaining off portion of each duty cycle period. The ratio of the on portion to the duty cycle period defines the duty cycle. For example, if the on portion is one half of the duty cycle period, the duty cycle would be 50%. In various embodiments, the stimulation can be set to be delivered at a duty cycle in the range greater than and/or less than one or more of 5%, 10%, 15%, 20%, 25%, 30%, 35%, 40%, 45%, 50%, 55%, 60%, 65%, 70%, 75%, 80%, 85%, 90%, 95%, or 100%. The period used to define the duty cycle may be 60 seconds such that a 50% duty cycle would comprise 30 seconds of pulsing and 30 seconds of quiescence in each duty cycle period. In some embodiments, to ensure preservation of the nerve, a duty cycle of 10% to 50% may be preferable. In some embodiments, duty cycles up to 100% may be useful in particular circumstances. Those of skill in the art will recognize that one or more of the above percentages can be used as a border of a range of duty cycles.

What is claim is:

1. A method for trigeminal nerve stimulation, comprising:
positioning an electrode template on a patient so that a first aperture in the electrode template is over a supraorbital nerve on one side of a patient's forehead and so that a second aperture in the electrode template is over a supraorbital nerve on an opposing side of the patient's forehead;
applying a first cutaneous electrode through the first aperture in the electrode template so that the first cutaneous electrode is cutaneously applied over the supraorbital nerve on the one side of a patient's forehead;
applying a second cutaneous electrode through the second aperture in the electrode template so that the second cutaneous electrode is cutaneously applied over the supraorbital nerve on the opposing side of the patient's forehead; and
driving a current between the first cutaneous electrode and the second cutaneous electrode at specified operational parameters to provide the trigeminal nerve stimulation.

2. The method of claim 1, wherein driving the current comprising driving the current at a frequency between approximately 20 and 300 Hertz, at an amplitude of 0.1 to 3 milliamperes (mA), and at a pulse duration of less than or equal to 500 microseconds.

3. The method of claim 1, wherein driving the current further provides a treatment of a neurological disorder for the patient.

4. The method of claim 1, wherein driving the current further provides a treatment of a neuropsychiatric disorder for the patient.

5. The method of claim 1, wherein driving the current further provides a treatment of a medical disorder for the patient.

6. A template, comprising:
a planar template body;
a first aperture at a first lateral edge of the template body; and
a second aperture at an opposing second lateral edge of the template body, wherein the template body is laterally elongated from the first lateral edge to the second lateral edge such that the first aperture is configured for cutaneous placement of a first electrode over a supraorbital nerve on one side of a patient's face forehead and such that the second aperture is configured for cutaneous placement of a second electrode comprising over a remaining supraorbital nerve on an opposing side of the patient's forehead.

7. The template of claim 6, further comprising an alignment feature on the template body midway between the first aperture and the second aperture.

8. The template of claim 7, wherein the alignment feature is chevron-shaped.
